(19) United States
(12) Patent Application Publication
(10) Pub. No.: US 2023/0390555 A1
(43) Pub. Date: Dec. 7, 2023

(54) MEDICAL DEVICE FOR PROVIDING RELIEF IN FASCIA-RELATED MUSCLE PAIN AND STIFFNESS

(71) Applicant: Jessica Stavale, Oxford, MI (US)

(72) Inventor: Jessica Stavale, Oxford, MI (US)

(21) Appl. No.: 18/328,948

(22) Filed: Jun. 5, 2023

Related U.S. Application Data
(60) Provisional application No. 63/348,549, filed on Jun. 3, 2022.

(51) Int. Cl. A61N 1/36; A61H 7/00

(52) U.S. Cl. CPC A61N 1/36021; A61H 7/005; A61H 2201/0207; A61H 2201/10; A61H 2201/1695

(57) ABSTRACT

A medical device for providing relief in unhealthy fascia-related medical conditions, such as pain and stiffness. The apparatus includes a handpiece and a probe for applying a combination of bipolar radiofrequency energy and pressure to the affected area. The device also includes a heating plate for heating the affected area to further enhance the efficiency of the medical device.

### CROSS-REFERENCE TO RELATED APPLICATIONS

This application claims priority from a U.S. Provisional Patent Application Ser. No. 63/348,549, filed on Jun. 3, 2022, the disclosure of which is incorporated herein by reference in its entirety.

### FIELD OF INVENTION

The present invention relates to a medical device, and more particularly, the present invention relates to a medical device for releasing fascia connective tissue adhesion and tightness.

### BACKGROUND

Fascia is a thin sheet of connective tissue that surrounds and holds every organ and muscle in place. Fascia provides a framework that supports and protects individual muscles, organs, and nerves. Fascia surrounds or runs through by being interconnected to every system of the body.

Unhealthy fascia can occur due to a variety of medical conditions that primarily result in fascia-related pain and stiffness. Fascia can become tight, twisted, or knotted, or form adhesions (restrictions), or become sticky, clumpy, flaky, and form distortions. Disorders of fascia, such as tightness, stiffness, adhesion, and the like can cause pain. Massaging the affected area is a widely practiced method for instant relief. A variety of electronic and mechanical massagers are available for massaging the affected areas; however, the known massagers are largely ineffective in providing relief in fascia-related medical conditions.

A need is therefore appreciated for a device for use in medical conditions, chiefly pain, associated with unhealthy fascia.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a medical device that can effectively provide relief in unhealthy fascia-related pain and other medical conditions.

It is another object of the present invention that the device be compact and easy to use.

It is a further object of the present invention that the device be economical to manufacture.

It is still a further object of the present invention that the device acts through multiple modes of action.

In one aspect, disclosed is a device that used bipolar radiofrequency and pressure to provide relief in medical conditions, including pain, associated with unhealthy fascia. The disclosed device may act by releasing the aberrated fascia layers to aid in restoring the health of fascia tissue volume.

In one aspect, disclosed is a method for providing relief in unhealthy fascia-related medical conditions, the method comprises providing a medical device; and applying, externally, through the medical device, over an affected area, a combination of bipolar radiofrequency energy and pressure. The medical device comprises a handpiece and a probe, the probe comprises a plurality of massage heads, wherein the pressure is applied by the plurality of massage heads. The plurality of massage heads are of an elongated profile. The medical device further comprises a heating plate surrounded by the plurality of massage heads, wherein the method further comprises applying, through the heating plate, heat to the affected area. The probe is interchangeably coupled to the handpiece. The medical device further comprises a battery. The medical conditions comprise pain and stiffness.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a perspective view of a medical device, according to an exemplary embodiment of the present invention.

FIG. 2 shows a probe of the medical device, according to an exemplary embodiment of the present invention.

FIG. 3 shows a top view of the probe of FIG. 2, according to an exemplary embodiment of the present invention.

FIG. 4 shows a side view of the probe of FIG. 2, according to an exemplary embodiment of the present invention.

FIG. 5 shows another embodiment of the probe that is smaller in size, according to the present invention.

### DETAILED DESCRIPTION

Subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as apparatus and methods of use thereof. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention since the scope of the invention will be best defined by the allowed claims of any resulting patent.

The following detailed description is described with reference to the drawings, wherein like reference numerals are used to refer to like elements throughout. In the following description, for purposes of explanation, specific details may be set forth in order to provide a thorough understanding of the subject innovation. It may be evident, however, that the claimed subject matter may be practiced without these specific details. In other instances, well-known structures and apparatus are shown in block diagram form in order to facilitate describing the subject innovation. Moreover, the drawings may not be to scale.

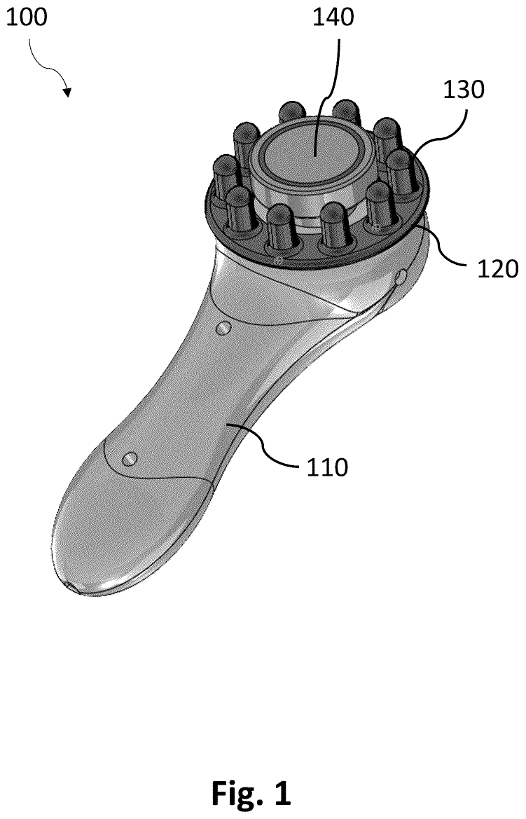

Disclosed is a medical device for use in medical conditions associated with unhealthy fascia. Particularly, the present invention can be used to provide relief from pain and stiffness associated with unhealthy fascia. The disclosed medical device can act through multiple modes of action to normalize the unhealthy fascia tissue layers. The disclosed device uses a combination of pressure and bipolar radiofrequency energy for the release of adhesion or tightness of fascia. The disclosed device can be used externally and noninvasively over an affected bodily volume to deliver pressure and bipolar radiofrequency energy. Bipolar radiofrequency typically refers to delivering high-intensity, high-frequency alternating current transfer to tissues, resulting in electrothermal reactions. Referring to FIG. 1, the disclosed medical device 100 includes a handpiece 110 and a probe 120 coupled to the handpiece. The handpiece can include a suitable mechanism for generating bipolar radiofrequency. Such mechanisms for the generation of bipolar radiofrequency are known in the art and any such mechanism is within the scope of the present invention. The disclosed medical device can have batteries to power the RF generation mechanism. For example, rechargeable batteries can be used, and a charging circuitry can also be provided to charge the batteries. Alternatively, the disclosed device can be powered by an external power supply. Preferably, the medical device can include a base unit that includes the batteries and charging circuitry, and the handpiece can be electrically connected to the base unit. The housing of the base unit can also be molded according to the shape of the handpiece so that the handpiece can be safely stored over the base unit. One or more components of the disclosed medical device can be disposed in either the handpiece or the base unit without departing from the scope of the present invention. Also, the components described herein as a part of, or included within the handpiece, can also be incorporated into the base unit, if suitable or possible. The base unit can be bigger in size than the handpiece and can be placed on a suitable surface while the operator can handle the handpiece to provide the therapy.

The housing of the handpiece can be ergonomically designed such that a user can hold the handpiece in hand and apply suitable pressure from the handpiece over the skin. The user may apply suitable pressure for a desired duration over the affected area.

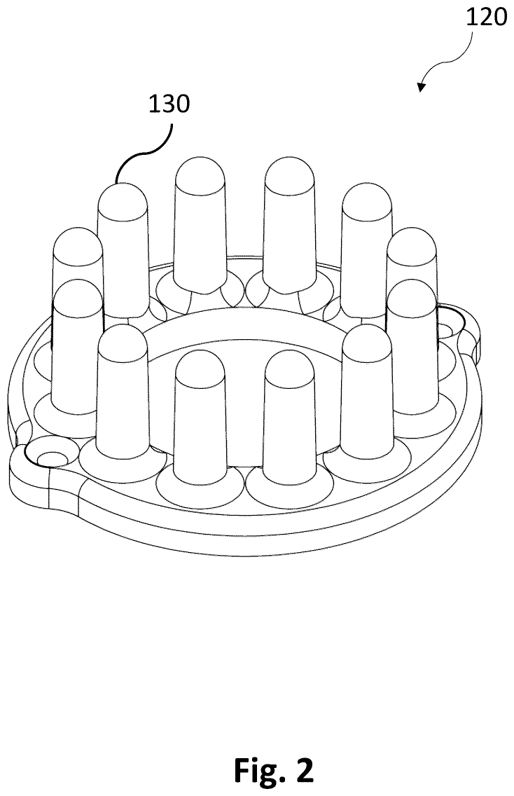

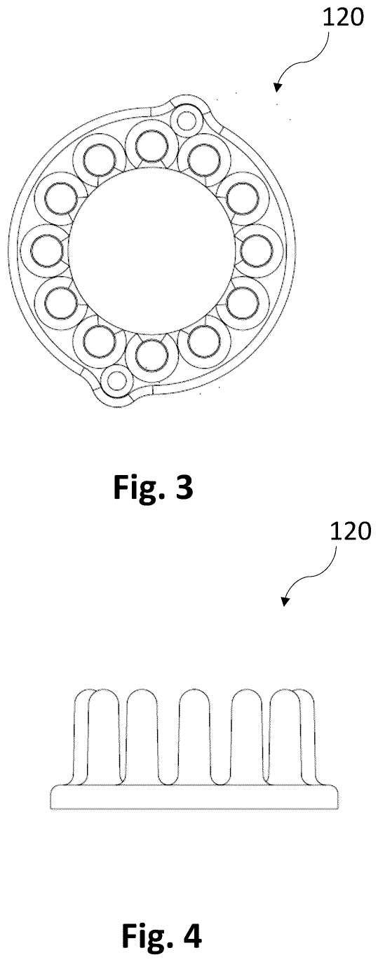

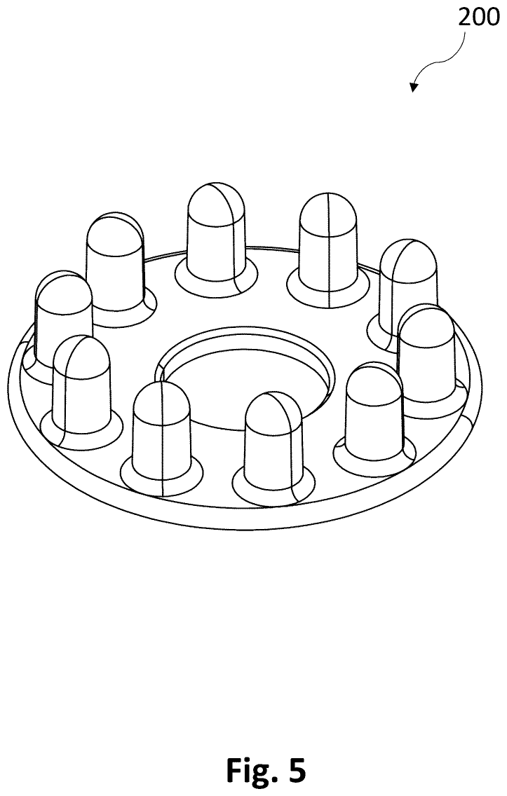

As shown in FIGS. 1-4, the probe head 120 can include multiple massage heads 130 arranged along a circle. The profile of the massage heads allows the application of deep pressure to the volume of the tissue. The novel design of the massage heads helps to release the superficial and deep layers of fascia, which also aids in providing a myofascial release and massage. The number of massage heads can vary, and the size of massage heads can be made smaller or larger, and any such modification is within the scope of the present invention. The smaller probe heads can be designed for the face, neck, and delicate areas, while the larger probe heads can be used over the rest of the body. FIG. 5 shows an example of the smaller probe head 200, that has about 10 massage heads. It is to be noted that the probe heads can be interchangeable, and any size probe head can be used with the handpiece.

The device can also include a heating plate 140 surrounded by massage heads. The casing of the probe head can be hollow at the center to accommodate the heating plate. The device can provide high-frequency deep heat in combination with the pressure applied by the probe that is designed for the application of pressure at a specific depth to the tissue, allowing the fascia to release as it heats up. The disclosed device can aid in restoring fascia to optimal health by allowing the hydration to return to the tissue from the combination of the radiofrequency and probe head to release any form of adhesion/restriction/distortion, so the fascia becomes smooth, slippery, and hydrated.

The medical device uses bipolar radiofrequency with a built-in temperature control chip with an output of 1.0 MHz. The power output can be up to 25 W for the face component and up to 50 W for the body component, with three intensity levels to choose from. A suitable interface can be provided for the user to select between different options, such as intensity levels. The medical device can also include a control unit/controller that can receive different settings and operate the device accordingly. The interface can be coupled to the control unit for input by the user. The interface can include hard buttons or soft buttons, including providing the interface on an external computing device, such as a smartphone. An on/off button can also be provided to allow the user to turn on and off the device. The user can also preset the duration of operation of the device, and such duration can be set through the interface. Such an interface can be provided in the base unit of the medical device.

The disclosed medical device can provide high-frequency deep waves in combination with an attached fascia probe head, designed at a specific depth that applies pressure to the tissue allowing the fascia to release. Additionally, a heating plate can also be used to provide external heat to the tissue volume.

The disclosed medical device can reduce fine lines, firm, and tighten the skin, stimulate collagen production, improve circulation, reduce cellulite, reduce tension, and aid in soothing sore muscles (they are encased in fascia and have fascia running through them). This is possible by restoring health to the fascia through the combination of the bipolar radiofrequency and pressure by the probe.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for providing relief in unhealthy fascia-related medical conditions, the method comprises:
providing a medical device; and
applying, externally, through the medical device, over an affected area, a combination of bipolar radiofrequency energy and pressure.

2. The method according to claim 1, wherein the medical device comprises a handpiece and a probe, the probe comprises a plurality of massage heads, wherein the pressure is applied by the plurality of massage heads.

3. The method according to claim 2, wherein the plurality of massage heads are of an elongated profile.

4. The method according to claim 2, wherein the medical device further comprises a heating plate surrounded by the plurality of massage heads, wherein the method further comprises:
applying, through the heating plate, heat to the affected area.

5. The method according to claim 2, wherein the probe is interchangeably coupled to the handpiece.

6. The method according to claim 2, wherein the medical device further comprises a battery encased within a base unit of the medical device, the handpiece electrically connected to the base unit.

7. The method according to claim 2, wherein the medical conditions comprise pain and stiffness.

8. A medical device for providing relief in unhealthy fascia-related medical conditions, the medical device comprises:
a handpiece; and
a probe mounted over the handpiece, the probe comprises a plurality of massage heads,
wherein the medical device is configured to apply, externally, over an affected area, a combination of bipolar radiofrequency energy and pressure.

9. The medical device according to claim 8, wherein the pressure is applied by the plurality of massage heads.

10. The medical device according to claim 9, wherein the plurality of massage heads are of an elongated profile.

11. The medical device according to claim 9, wherein the medical device further comprises:
a heating plate surrounded by the plurality of massage heads, the heating plate configured to apply heat to the affected area.

12. The medical device according to claim 9, wherein the probe is interchangeably coupled to the handpiece.

13. The medical device according to claim 9, wherein the medical device further comprises:
a battery encased within a base unit of the medical device, the handpiece electrically connected to the base unit.

14. The medical device according to claim 9, wherein the medical conditions comprise pain and stiffness.
